(19) United States
(12) Patent Application Publication
(10) Pub. No.: US 2012/0135485 A1
(43) Pub. Date: May 31, 2012

(54) SORBENT AND PROCESS FOR REMOVING FERMENTATION INHIBITORS

(75) Inventors: William J. Koros, Atlanta, GA (US); Kuang Zhang, Atlanta, GA (US)

(73) Assignee: Georgia Tech Research Corporation, Atlanta, GA (US)

(21) Appl. No.: 12/955,650

(22) Filed: Nov. 29, 2010

(51) Int. Cl. C12P 7/04; C12P 1/00; D21C 3/00; C08F 124/00; C08F 2/06

(52) U.S. Cl. 435/157; 526/270; 526/212; 162/72; 162/29; 435/41

(57) ABSTRACT

The invention pertains to, for example, an improved sorbent and process for removing fermentation inhibitors such as furfural and/or HMF in microbial processes utilizing fermentable sugars obtained from biomass including, for example, in the production of bioalcohols. The sorbent is capable of separating one or more inhibitors from monosaccharides and is characterized by: (1) a K_{sugar} partition coefficient of less than about 5 and (2) one or more of the following characteristics: (a) a furfural sorption capacity of at least about 200 mg/g sorbent at a furfural solution concentration of 2.5 grams per liter of water; (b) an Mt/M∞ of at least about 0.9 at 7.5 sec^{1/2}; and (c) a K_{furfural} partition coefficient of greater than about 3000.

### FIELD OF THE INVENTION

The instant invention pertains to, for example, an improved sorbent and process for removing fermentation inhibitors such as furfural and/or HMF in microbial processes utilizing fermentable sugars obtained from biomass including, for example, in the production of bioalcohols.

### BACKGROUND AND SUMMARY OF THE INVENTION

The enzymatic hydrolysis of cellulose to glucose has gained increased interest over the last ten years, and growing demand for economically sustainable biofuels points to an urgent need for making existing processes more efficient and also reducing costs in their production. Cellulose, a polysaccharide made by many plants, is one of the most abundant organic compounds on Earth and therefore represents a potential goldmine for the biofuel industry. That is, enzymatic hydrolysis may be employed to produce fermentable sugars such as glucose which may be feremented to biofuels like alcohols, fatty alcohols, hydrocabons, fatty acids, triglycerides, terpenes, and combinations thereof. Unfortunately, current enzymatic degradation of cellulose to fermentable sugars faces major issues that prevent its wide utilization.

For example, the process of ethanol production using biomass as a feedstock is well known (http://www.vermontbiofuels.org/biofuels/ethanol.shtml). In this process, both glucose and pentose are fermented to ethanol by a microorganism. Currently, yeast (Saccharomyces cerevisiae) is often used in the process, see, Almeida, J. R. M., et al., J. of chem. tech. and biotech., 2007, 82(4): p. 340-349. However, other microorganisms, for example, Zymomonas mobilis (Z. Mobilis) may also be used in the process.

Microorganisms such as Z. Mobilis used in various fermentation processes are often sensitive to various chemicals such as furfural and HMF which are often produced during hydrolysis of lignocellulosic biomass and may inhibit microbial growth. Various fermentation microbes are able to partially reduce these aldehydes to corresponding alcohols and thus partially reduce the toxicity associated with furfural and HMF. However, such detoxification processes may result in a significant lag phase or not even work with higher concentrations of furfural and HMF. For example, the presence of 7.3 mM furfural or 9.5 mM HMF can reduce the growth rate of Z. Mobilis by 25% while at a concentration of 52 mM furfural or 63 mM HMF, the growth is completely inhibited according to Franden, Pienkos, et al., “Development of a high-throughput method to evaluate the impact of inhibitory compounds from lignocellulosic hydrolysates on the growth of Zymomonas mobilis.” Journal of Biotechnology 144(4): 259-267. Therefore, it would be desirable to discover alternative methods and compositions which can assist in reducing furfural and/or HMF in microbial processes utilizing fermentable sugars obtained from biomass including, for example, in making bioethanol, which are effective and efficient.

Advantageously, the invention relates in one embodiment to a sorbent capable of separating one or more inhibitors from monosaccharides. The sorbent is characterized by a K_{sugar} partition coefficient of less than about 5. The sorbent is also characterized by one or more of the following characteristics: (a) a furfural sorption capacity of at least about 200 mg/g sorbent at a furfural solution concentration of 2.5 grams per liter of water; (b) an Mt/M∞ of at least about 0.9 at 7.5 sec^{1/2}; and (c) a K_{furfural} partition coefficient of greater than about 3000. The sorbent may made by a which comprises first polymerizing furfural to form a polyfurfural and then pyrolyzing the polyfurfural at a temperature of at least about 800° C. in a substantially inert atmosphere.

In another embodiment the invention relates to a process for treating a lignocellulosic feedstock composition comprising one or more sugars and furfural. The process comprises contacting the lignocellulosic feedstock with a sorbent under conditions such that the furfural in the composition is reduced to less than about 0.1 grams of furfural per liter of the total composition. As described previously the sorbent is characterized by a K_{sugar} partition coefficient of less than about 5. It also is characterized by ne or more of the following characteristics: (a) a furfural sorption capacity of at least about 200 mg/g sorbent at a furfural solution concentration of 2.5 grams per liter of water; (b) an Mt/M∞ of at least about 0.9 at 7.5 sec^{1/2}; and (c) a K_{furfural} partition coefficient of greater than about 3000.

### BRIEF DESCRIPTION OF THE FIGURES

FIG. 1 illustrates the shaker used to perform sorption tests in example 1.

FIG. 2 illustrates the sorption capacity and equilibrium time of sorbent in example 1.

FIG. 3 illustrates the monosaccharides analysis before and after the sorption test in example 1.

FIG. 4a illustrates cell growth during fermentation in example 1 wherein the dashed line represents fermentation without furfural and HMF and the solid line represents fermentation with 3.6 grams per liter furfural and HMF.

FIG. 4b illustrates sugar concentration and ethanol production during fermentation in example 1 wherein the dashed line represents fermentation without furfural and HMF and the solid line represents fermentation with 3.6 grams per liter furfural and HMF.

FIG. 5 illustrates cell growth during the fermentation with different content of furfural and HMF in example 1.

FIG. 6a illustrates sugar concentration with different content of furfural and HMF in example 1.

FIG. 6b illustrates ethanol production during the fermentation with different content of furfural and HMF in example 1.

FIG. 7 illustrates a regeneration cycle in column test of example 2.

FIG. 8 shows sorption capacity of Norit_{—}1240 in batch treatment.

FIGS. 9a and 9b show a regeneration cycle in column test and results using Norit_{—}1240 in example 2.

FIGS. 10a and 10b are SEM images of pyrolyzed polyfurfural useful in the present invention.

### DETAILED DESCRIPTION OF THE INVENTION

### Definitions

The term “fermentable sugar” refers to oligosaccharides and monosaccharides that can be used as a carbon source by, for example, a microorganism like Z. mobilis in a fermentation process.

The term “lignocellulosic” refers to a composition comprising both lignin and cellulose. Lignocellulosic material may also comprise hemicellulose and includes untreated biomass or treated biomass, e.g., biomass that has been treated in some manner prior to saccharification. Generally, biomass includes any cellulosic or lignocellulosic material and includes materials comprising cellulose, and optionally further comprising hemicellulose, lignin, starch, oligosaccharides and/or monosaccharides. Biomass may also comprise additional components, such as protein and/or lipid. Biomass may be derived from a single source, or biomass can comprise a mixture derived from more than one source; for example, biomass could comprise a mixture of corn cobs and corn stover, or a mixture of grass and leaves. Biomass includes, but is not limited to, bioenergy crops, agricultural residues, municipal solid waste, industrial solid waste, sludge from paper manufacture, yard waste, wood and forestry waste. Examples of biomass include, but are not limited to, corn grain, corn cobs, crop residues such as corn husks, corn stover, grasses, wheat, wheat straw, barley, barley straw, hay, rice straw, switchgrass, waste paper, sugar cane bagasse, sorghum, soy, components obtained from milling of grains, trees, branches, roots, leaves, wood chips, sawdust, shrubs and bushes, vegetables, fruits, flowers and animal manure.

The term “suitable fermentation conditions” refers to conditions that support the production of ethanol using, for example, a microorganism like a Z. mobilis strain. Such conditions may include suitable pH, nutrients and other medium components, temperature, atmosphere, and other environmental factors.

### Sorbent

The novel sorbent of the invention is capable of separating one or more inhibitors from monosaccharides and is usually defined by certain characteristics. First, the sorbent is characterized by an ability to be permeable, i.e., not sorb, monosaccharides. This is advantageous in that the sugars are desired for the fermentation process. In one embodiment, the K_{sugar} partition coefficient of the sorbent (as measured and calculated in Example 2 below) is less than about 5, or less than about 3; or less than about 1, or less than about 0.7, or less than about 0.5. In some embodiments, the sorbent is alternatively or additionally characterized by a glucose sorption capacity of less than about 5 mg/g sorbent at a glucose solution concentration of 20 grams per liter of water wherein the sorption capacity is calculated assuming a K_{sugar} partition coefficient of less than 0.5 and a sorbent true density of 2 kg/L.

Second, the sorbent is usually characterized by one, or two, or three or more of the following characteristics: (a) a furfural sorption capacity of at least about 200, or at least about 220, mg/g sorbent at a furfural solution concentration of 2.5 grams per liter of water; (b) an Mt/M∞ of at least about 0.9, or at least about 0.94, at 7.5 sec^{1/2}; and (c) a K_{furfural} partition coefficient of greater than about 3000.

The composition of the sorbent is not particularly critical so long as it exhibits the characteristics as described above. Suitable sorbent compositions may include, for example, a zeolite, a polystryrene, a pyrolyzed polyfurfural, or a mixture thereof. One particularly preferable sorbent for some applications may be pyrolyzed polyfurfural.

Suitable pyrolyzed polyfurfurals may be unsubstituted or substituted with any suitable substituent so long as it does not interfere with the characteristics making it suitable for use in the desired applications. The pyrolyzed polyfurfural may be made in any suitable manner. One useful method comprises first polymerizing furfural to form a polyfurfural. The polymerization may or may not employ one or more catalysts and/or solvents. If employing a catalyst, then an acidic catalyst such as an H₂SO₄ catalyst may be useful. In addition, a lower alkanol solvent such as C1-C6 alcohol(s) like ethanol may be employed.

Subsequent to or simultaneously with the polymerization, the polyfurfural is pyrolyzed, i.e., heated in the substantial absence of oxygen, at a temperature of at least about 800° C. Generally, the heating may be accomplished in any convenient manner but is preferably conducted in a substantially inert atmosphere like argon. The pyrolysis may be conducted under pressure and pressures of from about 1 torr to about 50 ton (vacuum), or from about 760 torr to about 1520 torr (purging inert gas) are often useful. Once formed the pyrolyzed polyfurfural comprise nearly pure carbon and are often generally spherical with a particle size ranging from about 1.5 to about 2.5 micrometers. The pyrolyzed polyfurfural particles are useful in many separation applications including, but not limited to, treatment of lignocellulosic feedstock compositions to remove inhibitors like furfural and/or HMF.

### Processes for Removing, for Example, Furfural

A particularly useful application for the above-described sorbents is in a process for treating a lignocellulosic feedstock composition. As described above the lignocellulosic feedstock usually comprises any biomass useful for alcohol production. Cellulose is the most common form of carbon in the biomass and may often account for from about 40% to about 60% by weight of the biomass, depending on the biomass source. It usually comprises a complex sugar polymer, or polysaccharide, made from the six-carbon sugar, glucose. Hemicellulose is also a major source of carbon in biomass, at levels of from about 20% to about 40% by weight. It is a complex polysaccharide made from a variety of five- and six-carbon sugars. Accordingly, typical compositions that are useful in the process comprise one or more sugars such as glucose mixed with one or more inhibitors such as furfural, HMF, and/or mixtures thereof.

While the process of the invention may be employed on to an appropriate treated or untreated lignocellulosic composition at any stage, it is usually more effective to apply the process to a treated lignocellulosic composition. That is, the furfural inhibitor is often generated when a biomass feedstock is treated with, for example, steam and an acid like weak sulfuric acid to assist in breaking down the biomass. That is, the biomass is hydrolyzed to convert carbohydrates to sugars which hydrolysis unfortunately also often produces the inhibitors. The mechanism of generating the furfural inhibitor is not particularly important but may be due in part to dehydration and conversion of pentose sugars. In any event, the lignocellulosic feedstock comprising one or more sugars such as glucose mixed with one or more inhibitors such as furfural, HMF, and/or mixtures that is useful in the process may also comprise other ingredients such as acids and the like that result from the prior treatments.

The lignocellulosic feedstock comprising one or more sugars such as glucose mixed with one or more inhibitors is contacted with a sorbent under conditions such that the furfural in the composition is reduced to less than about 0.1 grams of furfural per liter of the total composition. Such contact may also assist in reducing or removing HMF, benzaldehyde, and/or acetaldehyde which are also often cited as inhibitors. The conditions under which it is contacted may vary depending upon the precise composition and sorbent. Generally, typical conditions may include contacting them at ambient temperature and pressure and advantageously higher temperatures usually do not significantly affect sorption in this particular liquid phase.

The sorbent to be employed in the process is described above and preferably pyrolyzed polyfurfural. It can be employed either batchwise or continuously as in a column. It may be efficient to regenerate the sorbent regardless of how the process is applied. In such event, the sorbent is typically contacted with a regenerating media to desorb the furfural and/or other inhibitors. Such regenerating media varies with the type of sorbent and the precise composition of what is sorbed on it. For sorbents used in the present invention such as pyrolyzed polyfurfural, an alcohol such as ethanol may often be employed as the regenerating media. And in one embodiment, the ethanol employed in the regeneration may be ethanol that results from a subsequent fermentation of the remaining sugars after removing at least a portion of the furfural with the sorbent.

In an embodiment of the invention, a process may be employed which uses two different sorbents. The first sorbent is used to reduce furfural to less than about 1.0 grams of furfural per liter of the total composition. Then, the second sorbent which comprises pyrolyzed polyfurfural is employed to reduce the furfural to less than about 0.1 grams of furfural per liter of the total composition. In this manner perhaps a more economically efficient sorbent like an activated carbon such as Norit_{—}1240 or activated carbons available from, for example, Sigma can be employed as a rough cut of the furfural before employing the highly selective pyrolyzed polyfurfural.

Irrespective of whether one sorbent is employed or two or more sorbents are employed to reduce furfural and/or other inhibitors, the remaining one or more sugars like glucose, fructose, sucrose, xylose, arabinose, mannose or a mixtures thereof may be fermented using any convenient means to produce a desired product. Such products include, for example, biofuels like alcohols, fatty alcohols, hydrocabons, fatty acids, tryglycerides, terpenes, and combinations thereof. A particularly preferable product is an alcohol like ethanol. Suitable fermentation conditions are known in the art and may include both aerobic and anaerobic ferementation processes. Substrate concentrations of up to about 25% (based on glucose), and under some conditions even higher, may be used with ethanol producing microorganisms like yeast or Z. mobilis. Accordingly, the range of fermentation conditions may be quite broad. Likewise, any of the many known types of apparatus may be used for the production of desired products like ethanol by the process.

The fermentation process may be carried out as a batch process or parts or all of the entire process may be performed continuously. To retain the microorganisms in the fermenter, one may separate solid particles from the fluids. This may be performed by centrifugation, flocculation, sedimentation, filtration, etc. Alternatively, the microorganisms may be immobilized for retention in the fermenter or to provide easier separation.

In a certain embodiment, the process for utilizing the sugars to make, for example, ethanol, may be optimized by various techniques, including, but not limited to removal of other inhibitors, for example acetic acid, formic acid, 2-furaldehyde, 2-furoic acid, vanillin and hydroxybenzoic acid, from the pretreated biomass, finding more optimal fermentation conditions. Techniques for removal of acetic acid from the pretreated biomass include, but are not limited to, use of ion-exchange resins and ion exchange membranes. The fermentation conditions may be further improved by taking into consideration both biomass and sugar utilization when selecting the conditions as both may be factors.

After fermentation, the products, for example, ethanol, may be separated from the fermentation broth by any of the many conventional techniques known to separate such products like ethanol from aqueous solutions. These methods include evaporation, distillation, solvent extraction and membrane separation. Particles of substrate or microorganisms may be removed before separation to enhance separation efficiency.

Once the fermentation is complete, excess microorganisms and unfermented substrate may be either recycled or removed in whole or in part. If removed, the microorganisms may be killed, dried or otherwise treated. This mixture may then be used as animal feed, fertilizer, burnt as fuel or discarded.

### Example 1

### PF800 Sorbent Production and Selectivity in Batch System

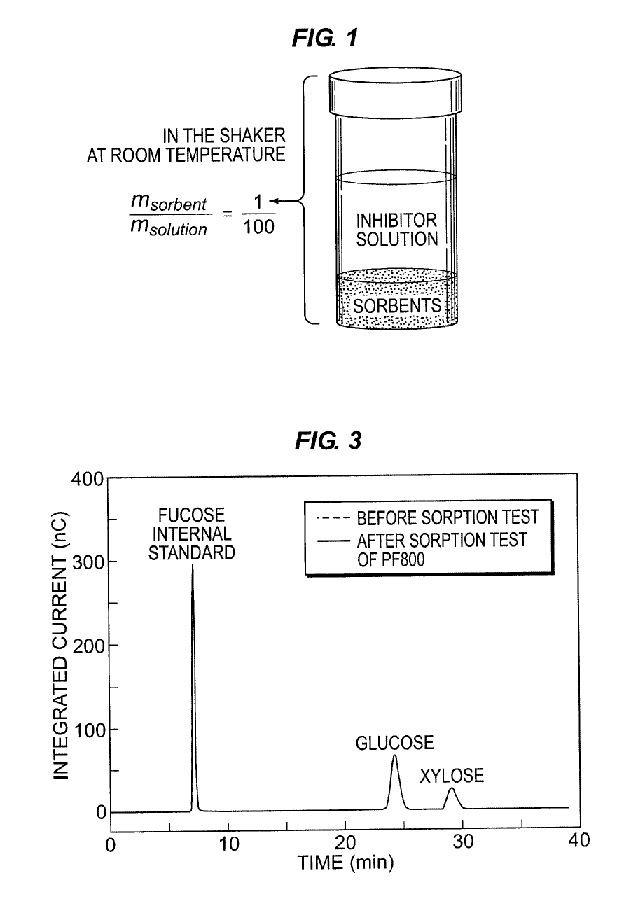

A sorbent (PF800) was made by synthesizing polyfurfural by using furfural as the only monomer, H2SO4 as the catalyst, and ethanol as the solvent. The polyfurfural was pyrolyzed at 800° C. under argon atmosphere. To investigate the sorption property of PF800, sorption tests were performed in a shaker under room temperature as shown in FIG. 1. The inhibitor solution had the following composition (the concentrations were chosen so as to be close to those in switch grass hydrolysate): 0.33 wt % furfural, 0.03 wt % HMF, 1.2 wt % glucose and 0.2 wt % xylose. The concentration of furfural & HMF in the solution was determined by UV-Vis spectrophotometer and HPLC, respectively.

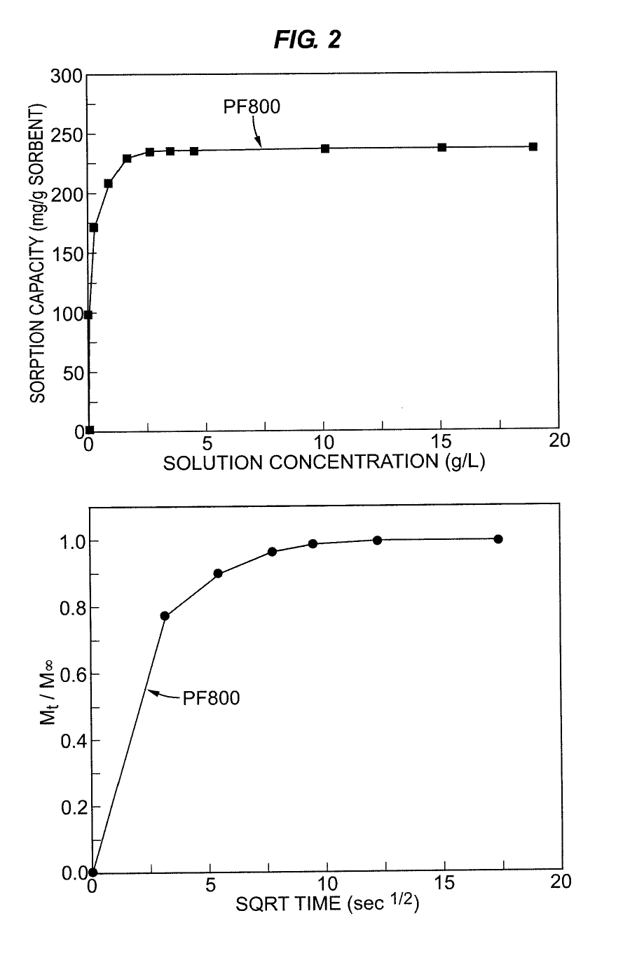

As shown in FIG. 2, the PF800 shows large sorption capacity even at low solution concentration. This indicates good affinity between PF800 and furfural. Additionally, PF800 reaches equilibrium quickly which is indicative of rapid mass transfer property. The large sorption capacity and rapid mass transfer property suggests that PF800 may be suitable for commercial applications.

Hydrolysates in the solution, such as glucose and xylose, are to be converted into ethanol in the fermentation, and ideally should not be removed during the separation. Otherwise, the overall wood-to-ethanol conversion would be diminished if sugars are being removed with the furfural. Therefore, the monosaccharide content in the solution was investigated before and after the sorption test by high performance anion exchange chromatography (HPAEC). As shown in FIG. 3, the peaks of monosaccharide superpose before and after the sorption test. This indicates that a significant amount of monosaccharides were not removed from the solution during the sorption test. This also confirms the highly selective separation of inhibitors by PF800.

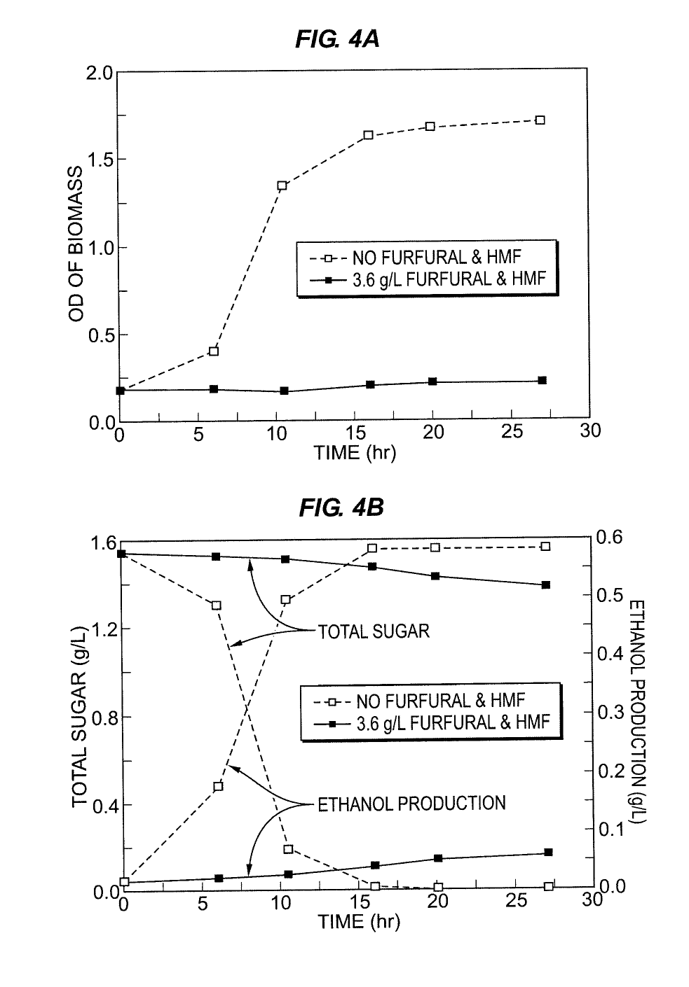

The influence of furfural and HMF on the fermentation is shown by the inhibition on cell growth of Z. mobilis A3. Optical cell density results are shown in FIG. 4(a). An exponential growth was observed when there is no furfural and HMF. As a comparison, when there were 3.6 grams per liter furfural and HMF in the broth, the cells almost stopped propagating during the fermentation. Consequently, sugar consumption and ethanol production were extremely slow as shown in FIG. 4(b). Therefore, furfural and HMF are strong inhibitors at the level of 3.6 grams per liter during the ethanol fennentation of Zymomonas mobilis A3.

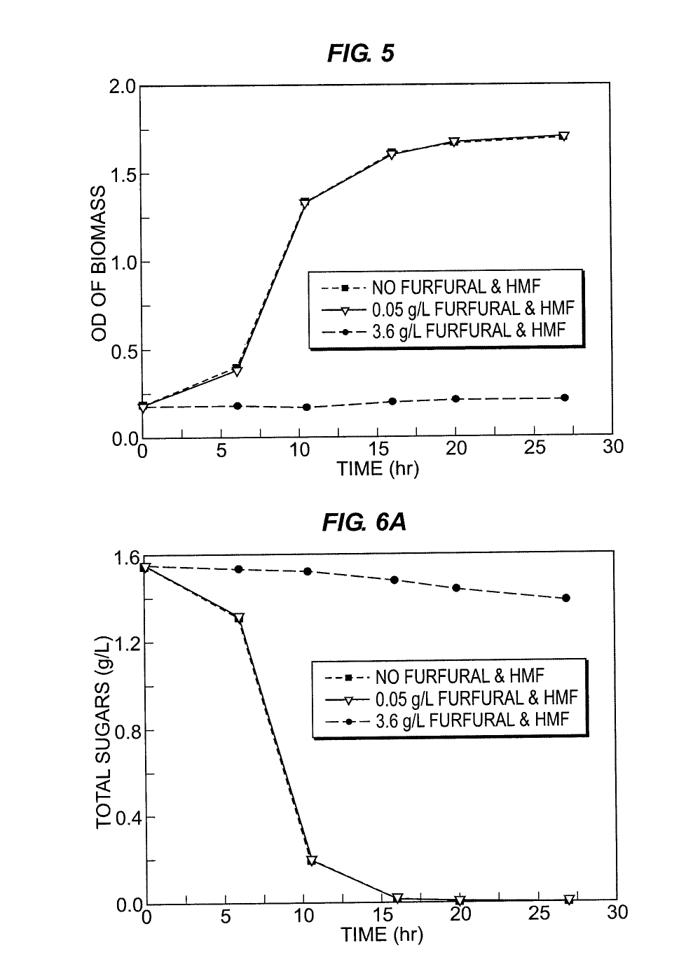

Next, the furfural and HMF are separated from the broth using the sorbent PF800 described above. The mass ratio of PF800 to broth was performed at 1/100 and resulted in 0.045 grams of furfural per liter and 0.005 grams of HMF per liter left in the broth after sorption. As shown in FIG. 5 the overlapping of the line pertaining to no furfural and HMF with the line pertaining to 0.05 grams of furfural plus HMF per liter indicates that cell growth completely recovers at 0.05 grams of furfural plus HMF per liter.

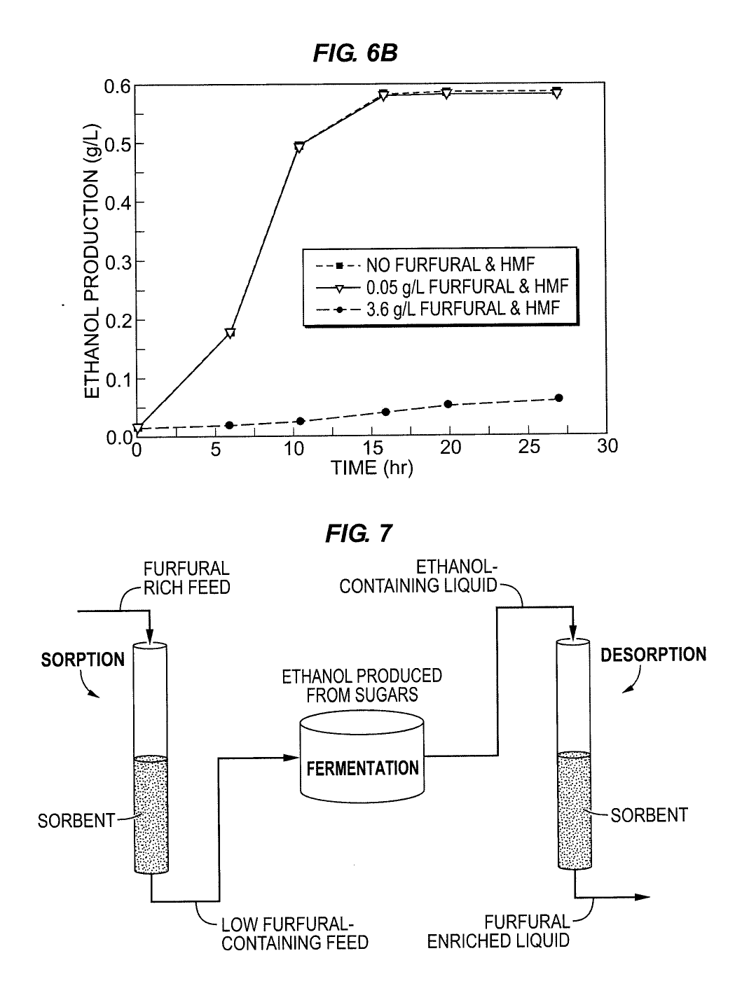

The sugar consumption and ethanol production were compared at various furfural and HMF concentrations. The results in FIG. 6 show the relationship between furfural and HMF concentration and cell growth. Both sugar consumption and ethanol production completely recover at 0.05 grams per liter furfural and HMF. No sugar loss was observed after sorption which was also indicated by no loss of ethanol production. This confirms the surprising and unexpected selectivity of the PF800 sorbent.

### Example 2

### PF800 and Norit 1240 Partition Coefficient and Sorption Test in Column System

PF800's K_{furfural}, partition coefficient of furfural shown below is calculated by the ratio of furfural concentration in sorbent to furfural concentration in liquid and is about 4100. PF800's K_{sugar}, partition coefficient of sugar shown below is calculated by the ratio of sugar concentration in sorbent to sugar concentration in liquid and in contrast to K_{furfural} is only about 0.56. The small value of K_{sugar} indicates PF800 hardly adsorbs sugar in the water solution. As for Norit_{—}1240, the commercial activated carbon from Norit company, K_{sugar} increases to 6.5, while K_{furfural} is 4200, similar to PF800's K_{furfural} Since K_{furfural} is still much larger than K_{sugar}, the affinity between Norit_{—}1240 and furfural is larger than the affinity between Norit_{—}1240 and sugar. As a result, Norit_{—}1240 adsorbs furfural preferentially to sugar in the water solution. However, when furfural concentration drops to a low level, Norit_{—}1240 begins to adsorb sugar at a considerable level.

$$PF 800 \text{:} \frac{{K}_{furfural}}{{K}_{sugar}} = \frac{{C}_{furfural\_carbon} / {C}_{furfural\_liquid}}{{C}_{sugar\_carbon} / {C}_{sugar\_liquid}} = \frac{4100}{0.56} = 7321$$

$$Norit \text{-} 1240 \text{:} \frac{{K}_{furfural}}{{K}_{sugar}} = \frac{{C}_{furfural\_carbon} / {C}_{furfural\_liquid}}{{C}_{sugar\_carbon} / {C}_{sugar\_liquid}} = \frac{4200}{6.5} = 646$$

A further experiment showed that Norit_{—}1240 does not adsorb sugar when there is 1 g/L furfural (and above) existing in the water. But when the furfural concentration falls below 1 g/L, Norit_{—}1240 adsorbs sugar. Moreover, the sorption capacity of Norit_{—}1240 for sugar can reach 120 mg/g, when there is no furfural in liquid. In comparison, PF800 does not adsorb sugar because of low K_{sugar}, even when there is only 0.1 g/L furfural. Thus, due to the difference of selectivity between Norit_{—}1240 and PF800 the two sorbents can be used in two steps to remove furfural from a water solution. The first step is to reduce furfural content from about 4 g/L, the concentration after biomass pretreatment, to about 1 g/L by using Norit_{—}1240, because the commercial carbon does not adsorb sugar in this range of furfural concentration. The second step is to use PF800 to decrease the furfural content from about 1 g/L to about 0.1 g/L, because the toxic effect of furfural is negligible at about 0.1 g/L. As a consequence, the majority of furfural (about 75%) would be removed by the commercial carbon, thereby potentially reducing the cost of sorbent as a whole and increasing the efficiency.

A sorption test in a column system was investigated. Low ethanol-containing water solution was found to desorb furfural from the sorbent. Thus, a sorption-desorption regeneration cycle was designed as shown in FIG. 7. After biomass pretreatment, a furfural-rich feed goes to a sorption column to remove furfural from liquid, followed by the low furfural-containing feed from sorption column going to fermentation to produce ethanol from the sugars. After fermentation, the ethanol-containing liquid flows back into the column to desorb the furfural from sorbent. Furfural enriched liquid then goes to distillation to purify ethanol from the solution. After desorption, the regenerated sorbent is ready for the next cycle of sorption-desorption. The efficiency of sorbent use is greatly improved by this regeneration method.

Norit_{—}1240 was studied in the column system. Norit_{—}1240 is granular (8-20 mesh) and suitable in s column system. During the sorption-desorption cycle, the “working sorption capacity” of Norit_{—}1240 for furfural is about 50 mg/g. This is the difference of sorption capacity of the sorption tests with and without 7.5% ethanol in the liquid as shown in FIG. 8. Therefore, the mass ratio of sorbent to water solution becomes 1/10 in column system, instead of 1/50 as in the batch treatment of Example 1.

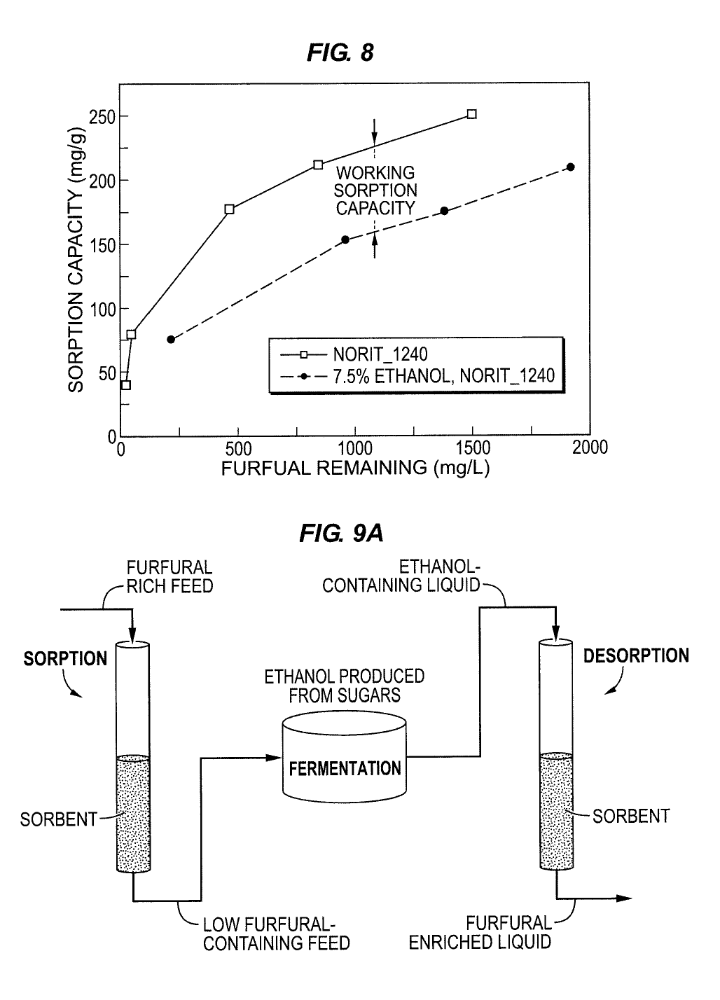

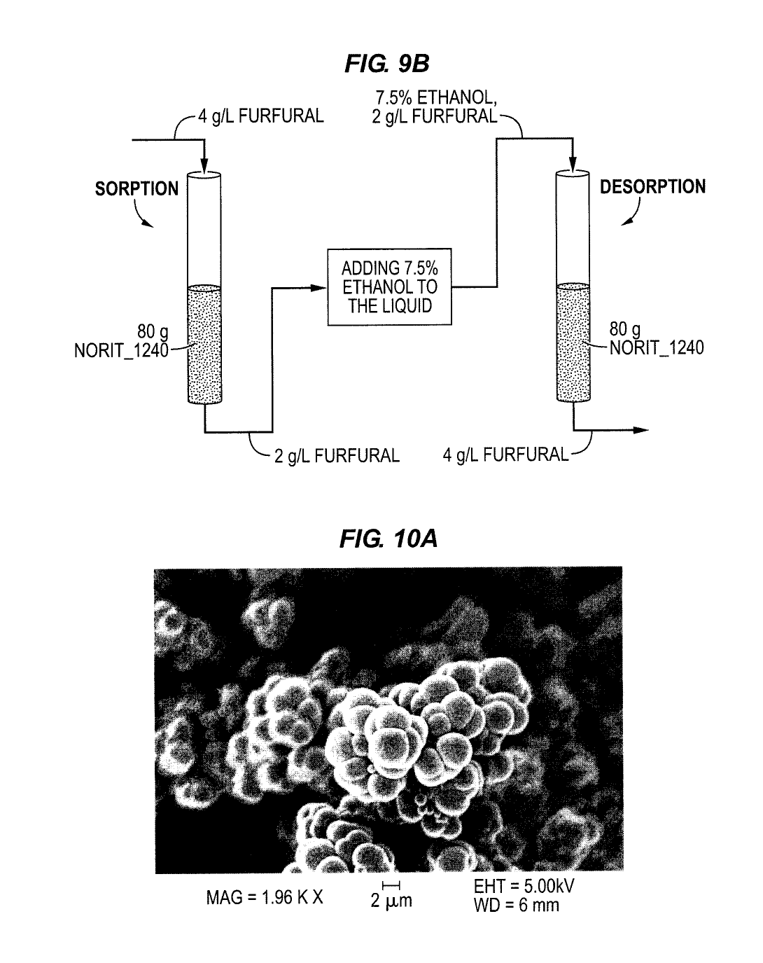

The step of fermentation between sorption and desorption in FIG. 7 was simulated by adding 7.5% ethanol into the liquid after sorption to simulate 7.5% ethanol produced in fermentation. The biomass-pretreated liquid, used in the sorption test, was simulated by the water solution with 4 g/L furfural, 2% glucose and 1% xylose. 80 g of Norit-1240 was placed in the column, while 800 ml water solution was pumped into the column, according to the mass ratio of 1/10. The flow rate was controlled at 50 ml/min. The procedure and results of the column test are shown in FIGS. 9a and 9b. After sorption, the furfural concentration was reduced from 4 g/L to 2 g/L. Meanwhile, the sugars were not removed through analyzing the glucose and xylose content by HPLC. And after desorption with 7.5%-ethanol-containing liquid, furfural concentration was enriched to 4 g/L again. Then, 800 ml fresh water solution with 4 g/L furfural, 2% glucose and 1% xylose was pumped into the column to start the next cycle of sorption and desorption test. The results were stable when the sorption-desorption cycle was run 20 times.

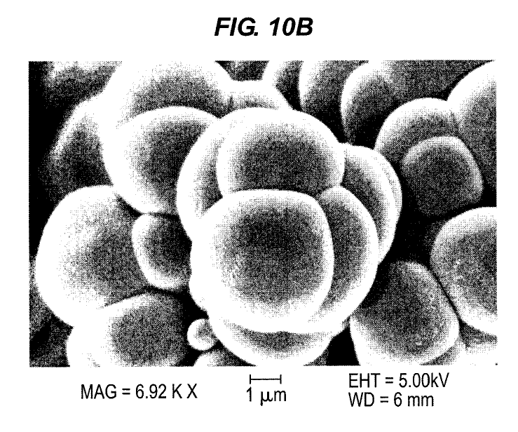

FIGS. 10a and 10b show SEM images of pyrolyzed polyfurfural useful in the present invention. As shown in the figures, the particles are generally spherical and may range in particle size from about 1.5 to about 2.5 micrometers.

The claimed subject matter is not to be limited in scope by the specific embodiments described herein. Indeed, various modifications of the invention in addition to those described herein will become apparent to those skilled in the art from the foregoing description. Such modifications are intended to fall within the scope of the appended claims.

All references cited herein are incorporated herein by reference in their entirety to the extent that they are not inconsistent and for all purposes to the same extent as if each individual publication, patent or patent application was specifically and individually indicated to be incorporated by reference in its entirety for all purposes.

The citation of any publication is for its disclosure prior to the filing date and should not be construed as an admission that the present invention is not entitled to antedate such publication by virtue of prior invention.

What is claimed is:

1. A sorbent capable of separating one or more inhibitors from monosaccharides wherein the sorbent is characterized by:
(1) a K_{sugar} partition coefficient of less than about 5; and
(2) one or more of the following characteristics: (a) a furfural sorption capacity of at least about 200 mg/g sorbent at a furfural solution concentration of 2.5 grams per liter of water; (b) an Mt/M∞ of at least about 0.9 at 7.5 sec^{1/2}; and (c) a K_{furfural} partition coefficient of greater than about 3000.

2. The sorbent of claim 1 wherein the K_{sugar} partition coefficient is less than about 1.

3. The sorbent of claim 1 wherein the K_{sugar} partition coefficient is less than about 0.7.

4. A sorbent of claim 1 which is characterized by two or more of the following characteristics: (a) a furfural sorption capacity of at least about 200 mg/g sorbent at a furfural solution concentration of 2.5 grams per liter of water; (b) an Mt/M∞ of at least about 0.9 at 7.5 sec^{1/2}; and (c) a K_{furfural} partition coefficient of greater than about 3000.

5. A sorbent of claim 1 which is characterized by all of the following characteristics: (a) a furfural sorption capacity of at least about 200 mg/g sorbent at a furfural solution concentration of 2.5 grams per liter of water; (b) an Mt/M∞ of at least about 0.9 at 7.5 sec^{1/2}; and (c) a K_{furfural} partition coefficient of greater than about 3000.

6. The sorbent of claim 1 wherein the sorbent comprises a zeolite, a polystryrene, a pyrolyzed polyfurfural, or a mixture thereof.

7. The sorbent of claim 1 wherein the sorbent comprises pyrolyzed polyfurfural which has been pyrolyzed at a temperature of at least about 800° C. in a substantially inert atmosphere.

8. A process of making a sorbent comprising (a) polymerizing furfural to form a polyfurfural; and (b) pyrolyzing the polyfurfural at a temperature of at least about 800° C. in a substantially inert atmosphere to form the sorbent.

9. The process of claim 8 wherein the polyfurfural is polymerized in the presence of an H₂SO₄ catalyst and a lower alkanol solvent.

10. The process of claim 8 wherein the polyfurfural is pyrolyzed in an argon atmosphere.

11. A process for treating a lignocellulosic feedstock composition comprising one or more sugars and furfural wherein the process comprises:
contacting the lignocellulosic feedstock with a sorbent under conditions such that the furfural in the composition is reduced to less than about 0.1 grams of furfural per liter of the total composition;
wherein the sorbent is characterized by:
(1) a K_{sugar} partition coefficient of less than about 5; and
(2) one or more of the following characteristics: (a) a furfural sorption capacity of at least about 200 mg/g sorbent at a furfural solution concentration of 2.5 grams per liter of water; (b) an Mt/M∞ of at least about 0.9 at 7.5 sec^{1/2}; and (c) a K_{furfural} partition coefficient of greater than about 3000.

12. The process of claim 11 wherein the process further comprises regenerating the sorbent after contacting the sorbent with the lignocellulosic feedstock.

13. The process of claim 12 wherein said regenerating comprises exposing said sorbent to a solution comprising an alcohol.

14. The process of claim 13 wherein said alcohol comprises ethanol.

15. The process of claim 11 wherein said sorbent comprises pyrolyzed polyfurfural.

16. The process of claim 11 wherein the contacting comprises first contacting the lignocellulosic feedstock with a first sorbent under conditions to form a first composition comprising less than about 1.0 grams of furfural per liter of the total composition; and then contacting said first composition with a second sorbent comprising pyrolyzed polyfurfural under conditions such that the furfural in the first composition is reduced to less than about 0.1 grams of furfural per liter of the total composition.

17. The process of claim 11 wherein the contacting the lignocellulosic feedstock with a sorbent reduces the concentration of one or more of the compounds selected from the group consisting of HMF, benzaldehyde, and acetaldehyde.

18. The process of claim 11 wherein the lignocellulosic feedstock is hydrolyzed prior to contacting it with the sorbent.

19. The process of claim 11 further comprising fermenting one or more sugars of the lignocellulosic feedstock composition subsequent to contacting the composition with said sorbent.

20. The process of claim 11 further comprising fermenting one or more sugars of the lignocellulosic feedstock composition to produce an alcohol subsequent to contacting the composition with said sorbent.

21. The process of claim 20 further comprising regenerating the sorbent by exposing said sorbent to at least a portion of the alcohol produced by fermenting.

22. The process of claim 11 wherein the sorbent is characterized by a K_{sugar} partition coefficient is less than about 1.

23. The process of claim 11 wherein the sorbent is characterized by a K_{sugar} partition coefficient is less than about 0.7.

24. The process of claim 11 wherein the sorbent is characterized by two or more of the following characteristics: (a) a furfural sorption capacity of at least about 200 mg/g sorbent at a furfural solution concentration of 2.5 grams per liter of water; (b) an Mt/M∞ of at least about 0.9 at 7.5 sec^{1/2}; and (c) a K_{furfural} partition coefficient of greater than about 3000.

25. The process of claim 11 wherein the sorbent is characterized by all of the following characteristics: (a) a furfural sorption capacity of at least about 200 mg/g sorbent at a furfural solution concentration of 2.5 grams per liter of water; (b) an Mt/M∞ of at least about 0.9 at 7.5 sec^{1/2}; and (c) a K_{furfural} partition coefficient of greater than about 3000.
